(19) United States
(12) Patent Application Publication
(10) Pub. No.: US 2023/0158091 A1
(43) Pub. Date: May 25, 2023

(54) CANNABINOID MEDICAL CARRIER

(71) Applicant: New York Medical College, Valhalla, NY (US)

(72) Inventors: Linda K. FRIEDMAN, Valley Stream, NY (US); Richard J. ZEMAN, New York, NY (US)

(21) Appl. No.: 17/912,278

(22) PCT Filed: Mar. 19, 2021
(86) PCT No.: PCT/US21/23093

§ 371 Date: Sep. 16, 2022

Related U.S. Application Data
(60) Provisional application No. 62/991,769, filed on Mar. 19, 2020.

(51) Int. Cl. A61K 36/185; A61K 47/42; A61K 47/36; A61K 47/28; A61K 9/00

(52) U.S. Cl. CPC A61K 36/185; A61K 47/42; A61K 47/36; A61K 47/28; A61K 9/0085; A61L 26/0066

(57) ABSTRACT

The present invention is generally directed to cannabinoid medical carriers that are effective in the treatment of internal wounds, such as brain or spinal cord injury. One embodiment comprises a application device consisting of a dissolvable biological carrier that contains a cannabinoid, which is readily adsorbed in vivo and is capable of conforming to the size and shape of the surface of the contusion site of a neurological trauma. The various elements of the medical carrier can be saturated with cannabidiol or other phytocannabinoids by diffusion under sterile conditions to achieve medicinal concentrations. The carrier can be applied through open or semi-open surgical procedures, or epidural or by subarachnoid/intrathecal delivery.

### FIELD OF THE INVENTION

The present invention provides a method and system of a medical carrier saturated with a cannabinoid, such as cannabidiol (CBD), or a combination of phytocannabinoids applied directly to an internal contusion site following injury, such as brain or spinal cord injury. Application can occur using open or semi-closed surgical procedures, or by epidural or subarachnoid/intrathecal delivery. The invention can prevent over-stimulation of acute inflammatory processes that follow a neurological trauma as well as prevent subsequent secondary neurodegeneration, paralysis, and potential onset to post-traumatic epilepsy. The invention can also be used with other anti-inflammatory/anti-oxidant substances (e.g. steroids, ascorbate), as well as on external wounds.

### BACKGROUND

Conventional methods and systems for current state of the art treatment of brain or spinal cord trauma include acute surgery and administration of steroids, and/or anti-epileptic drugs. The current treatment methods only provide limited effectiveness and have many undesirable side effects.

Following a neuro-trauma, the brain or spinal cord is susceptible to primary and secondary injury as a consequence of different biological mechanisms. Current state of the art treatment protocols only address a subset of issues that occur during the initial trauma. Moreover, oral administration and injection of treatment drugs are currently the most convenient and popular usages of drug/medication delivery. Administration of drugs, such as high-dose methylprednisolone, is used as a standard of care for acute medical treatment following a spinal cord injury. However, the existing methods suffer many drawbacks. For example, using high doses has adverse side-effects and should not be maintained over an extended period. After a head trauma, anticonvulsant drugs can be administered peripherally and acutely but may not actually treat or prevent posttraumatic neurological disorders, such as epilepsy (i.e. spontaneous seizures) that typically takes weeks or even months to develop. Thus, there is no safe treatment that addresses both acute and long-term therapeutic intervention that can universally treat acute neuro-trauma without adverse side-effects and have long-term benefit.

There is currently no standard of care to reduce immediate inflammation, reduce glutamate excitotoxicity, and preserve tissue for any neuro-traumatic event and/or prevent the onset of post-traumatic seizures associated with traumatic brain injury.

Cannabinoids are known chemical substances derived from the cannabis plant. CBD is a major non-psychoactive constituent of the plant. Nonintoxicating cannabinoids, such as CBD, are known to have therapeutic effects such as treatment of pain and nausea and vomiting. Currently, the common uses of cannabinoid treatments are topical, oral, or via smoking or vaping. Cannabinoids like CBD are lipophilic and therefore extracted from the cannabis plant as an oil.

A current problem with CBD treatments is how to administer CBD so it is readily absorbed by the body, and targeted to the site of trauma. One common administration method is through ingestion, where CBD is partially broken down by the liver and the digestive track, requiring numerous hours to take effect, with 5-20% absorbed in the body, untargeted. Another common administration method is through topical application, where CBD is infused into lotions, balms, creams, salves, and transdermal patches. Topicals provide concentrated relief to a particular area, however treatment is focused externally and does not treat internal wounds. A last common administration method is through vaping or smoking, where CBD directly enters the bloodstream within minutes, with 30-60% absorbed in the body, untargeted.

### BRIEF SUMMARY OF THE INVENTION

The present invention is a novel medical carrier such as bioabsorbable bandages or pellets or nanoparticles, saturated with cannabinoids, such as CBD, applied internally to a wound site to improve injury, such as neurotrauma. Direct dispensing of cannabinoids to the internal wound can reduce inflammation and neuronal impulses, and reduce tissue loss and spontaneous seizures, without adverse side-effects. The provided methods disclosed herein can be configured to contact a portion of the brain or spinal cord surface that has been injured by alternative surgical or intrathecal/epidural/subarachnoid methods of delivery. Since contusion sites vary in size and shape, a novel feature of the medical carrier its ability to adjust and accommodate the surface area affected.

The novel medical carrier device contains CBD or other cannabinoids and is configured to the size and shape of the injury site, and thus can be surgically applied internally and directly to the surface of the internal wound of injured neurological tissue. The medical carrier device disclosed herein can provide sustained but safe treatment at the contusion site over a long period and is bioabsorbable, having the advantage that it will eventually dissolve in vivo. The medical carrier device disclosed herein can also be used in conjunction with peripheral cannabinoid treatment by oral or subcutaneous administration.

The present disclosure describes an effective method to treat and suppress an immune response that causes neurological diseases but with decreased adverse side-effects. This method by single or dual use can also prevent epileptic activity of the brain and attenuate or prevent long-term paralysis.

The present invention provides a novel treatment strategy for a safe drug delivery and positioning system capable of reducing acute inflammation and excessive excitatory impulses that lead to acute neuronal cell death, secondary injury, and post traumatic epilepsy.

The present invention provides a bio-absorbable cannabinoid medical carrier that is able to accommodate different widths and concentrations of the cannabinoid, and can optionally be mixed with other anti-inflammatory/anti-oxidant substances. Moreover, the present disclosure describes pliability to configure the medical carrier device to the shape and size of the contusion site, whether it be open or closed.

The present invention provides an application device consisting of a dissolvable medical carrier containing at least one cannabinoid. In preferred embodiments, the carrier can be configured to different shapes and sizes, ranging from 0.1-500 mm x 0.1-500 mm, and readily adsorbs cannabinoids, such as CBD, and is capable of conforming to the size of the surface of the contusion site of a neurological trauma. In preferred embodiments, CBD, other phytocannabinoids, or other anti-inflammatory/anti-oxidant substances will be readily dissolvable within various biological compatible carriers and packaged under sterile conditions for widespread use in places such as hospitals. According to various embodiments, the medical device includes CBD at desired concentration, a shapeable carrier (e.g. gelatin sponge, cholesterol pellet, polysaccharide, polyglycholide, dissolvable silk, seaweed, two clay nanoparticles). In some embodiments, the medical carrier includes synthetic polymers such as polyglycolic acid, polylactic acid, polydioxanone, caprolactone, polyglactin, poliglecaprone. In some embodiments, the medical carrier is injectable as an injectable bandage. In some embodiments, the medical carrier is made from food, jelly-like substance, seaweed, and/or nanoparticles. In some embodiments, the medical carrier device can be semi-rigid, non-rigid, or of an appropriate viscosity. In some embodiments, the device further comprises sterile packaging for penetrating and non-penetrating contusions. In contrast to transdermal CBD treatment used for pain, cleansing, and moisturizing, the present invention disclosure of a medical carrier containing cannabinoids, such as CBD, allows direct application of the drug internally to the surface of a contusion site. The invention disclosed herein, including methods and systems, has the advantage of post-surgical dissolving in vivo which will prevent invading cells from hindering anti-inflammatory and regenerative processes, and will not necessitate subsequent surgical removal. Sustained local internal treatment of cannabinoids can heal the injured region before debilitating events predominate which will improve quality of life for neurological traumas. Sterile packaging of the application device will provide immediate availability for surgical use to acutely treat a neuro-trauma. The medical carrier can be interchanged and the purity of and/or the concentration of cannabinoids, such as CBD, or another anti-inflammatory/anti-oxidant substances, can also be changed for maximal efficacy of the drug and delivery system.

### BRIEF DESCRIPTION OF THE DRAWINGS

Some embodiments of the present invention are illustrated as an example and are not limited by the figures of the accompanying drawings, in which like references may indicate similar elements and in which:

FIG. 1A depicts one embodiment of a device comprising a medical carrier and at least one cannabinoid, wherein the medical carrier is saturated with the cannabinoid.

FIG. 1B depicts one embodiment of a medical carrier saturated with at least one cannabinoid applied directly to the surface of an internal injury site in a user's tissue. FIG. 1C depicts another embodiment of a medical carrier saturated with at least one cannabinoid applied directly to the surface of an internal injury site in a user's tissue, where the medical carrier conforms to the shape of the injury site.

FIG. 2A depicts a delivery agent containing a medical carrier saturated with at least one cannabinoid. FIG. 2B depicts one embodiment of a delivery agent wherein the medical carrier are pellets or particles, such as nanoparticles.

FIG. 3 depicts a method of treating an injury in a user's tissue with a device comprising a medical carrier and at least one cannabinoid, showing a primary injury site, secondary injury site(s), and treatment with direct application of a medical carrier saturated with at least one cannabinoid to the injury site(s).

FIG. 4 depicts one embodiment of obtaining a solution of at least one cannabinoid from a nearly pure form by diluting the solution with a carrier oil stepwise until an effective concentration is reached.

FIGS. 5A-H and 6A-E depict neuron and myelin-stained images of brain and spinal cord sections in rats after brain and spinal cord traumatic injury respectively, in presence and absence of a CBD medical carrier. Lesion sizes were reduced when a gelatin sponge saturated with CBD was administered. The injury epicenters were smaller after treatment with a CBD medical carrier. After direct treatment with a gelatin sponge saturated with CBD, the lesion areas were reduced in size at all injured levels. This was associated with significantly improved locomotion.

FIGS. 51 and 6F depict a weight drop model, where a CBD medical carrier significantly improved vestibulomotor function after brain injury in under 2 weeks' time. Motor functional recovery after spinal cord injury was observed after 4 weeks. After treatment with a CBD medical carrier, the time course of motor recovery improved and lesion size after spinal cord contusion was reduced.

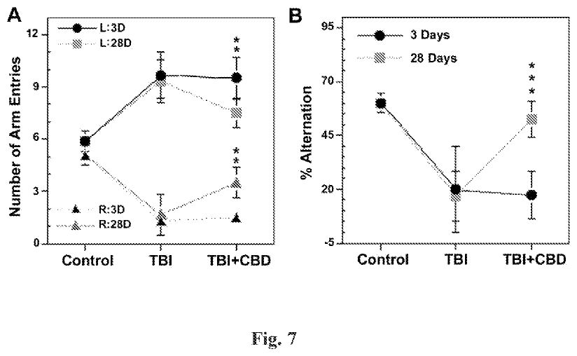

FIG. 7 depicts spontaneous alternating in a T-maze at 3 and 28 days following unilateral brain injury for assessment of spatial sense, memory, and cognitive ability in presence and absence of CBD. Naïve untreated rats spontaneously alternated between left and right arms. At 3 days, untreated and treated animals had marked asymmetry entering left. After 28 days, the asymmetry was reduced for CBD treated animals with a significant increase in percent alternating, whereas TBI rat's spontaneous alteration rates were unchanged.

### DETAILED DESCRIPTION OF THE INVENTION

The terminology used herein is for the purpose of describing particular embodiments only and is not intended to be limiting of the methods, compositions and systems disclosed herein. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined all terms including technical and scientific will have the same meaning as commonly understood by one having ordinary skill in the art to which this invention disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these have individual benefit and can be used in conjunction with one or more phytocannabinoids, or other anti-inflammatory/anti-oxidant agents. Accordingly, for the sake of clarity, this description will refrain from repeating every possible combination of the individual steps in an unnecessary fashion. Nevertheless, the specification and claims should be read with the understanding that such combinations are entirely within the scope of the invention provided carrier devices, methods and systems disclosed herein as well as the claims.

New application devices, systems, and methods comprising a medical carrier containing at least one cannabinoid, such as CBD, for internal delivery to a contusion or injury site of a user's tissue, such as brain or spinal cord, are discussed herein. In the following description, for purposes of explanation, numerous specific details are set forth in order to provide a thorough understanding of the present invention.

The present disclosure is to be considered as an exemplification of the invention and is not intended to limit the invention to the specific embodiments illustrated by the figures of description below.

In some embodiments, the present invention is a device comprising at least one cannabinoid and a medical carrier. In some embodiments, the cannabinoid is CBD. In some embodiments, the at least one cannabinoid is CBD and another cannabinoid. In some embodiments, the at least one cannabinoid is CBD and multiple other cannabinoids.

In some embodiments, the medical carrier is semi-rigid or non-rigid. In some embodiments, the medical carrier comprises a gelatin foam sponge. In some embodiments, the medical carrier comprises seaweed and nanoparticles. In some embodiments, the medical carrier comprises steroids. In some embodiments, the medical carrier comprises hydrogel. In some embodiments, the medical carrier comprises at least one of gelatin, polysaccharide, polyglycolide, glycerol, or cholesterol. In some embodiments, the medical carrier is configured to a size of 0.1-500 mm. In some embodiments, the medical carrier is configured to a size of 1-160 mm. In some embodiments, the medical carrier is configured to a size of 1-50 mm. In some embodiments, the medical carrier is configured to a size of 50-160 mm. In some embodiments, the medical carrier conforms to an injury site. In some embodiments, the medical carrier is configured to the shape of the injury site before application. In some embodiments, the medical carrier conforms to the shape of the injury site after application.

In some embodiments, the medical carrier contains CBD. In some embodiments, the medical carrier is saturated with CBD. In some embodiments, the medical carrier is saturated with 0.1-500 mg/ml of CBD. In some embodiments, the medical carrier is saturated with 1-10 mg/ml of CBD. In some embodiments, the medical carrier is saturated with 1-100 mg/ml of CBD. In some embodiments, the medical carrier is saturated with 100-500 mg/ml of CBD.

In some embodiments, the medical carrier saturated with CBD is dissolvable in vivo. In some embodiments, the medical carrier saturated with CBD is dissolvable in less than 24 hours. In some embodiments, the medical carrier saturated with CBD is dissolvable in less than 1 hour. In some embodiments, the medical carrier saturated with CBD is dissolvable in 1-3 days. In some embodiments, the medical carrier saturated with CBD is dissolvable in 5-7 days. In some embodiments, the medical carrier saturated with CBD is dissolvable in 14 days. In some embodiments, the medical carrier saturated with CBD is dissolvable in 14-30 days.

In some embodiments, the medical carrier device saturated with CBD is applied to an injury site. In some embodiments, the application is direct. In some embodiments, the injury site is internal. In some embodiments, the injury site is the brain. In some embodiments, the injury site is the spinal cord. In some embodiments, the injury site follows a neurological trauma. In some embodiments, the injury site follows a closed brain injury. In some embodiments, the injury site follows a penetrating brain injury. In some embodiments, the injury site follows a closed spinal cord injury. In some embodiments, the invention treats neurological trauma resulting from at least one neurological disorder including, post-traumatic epilepsy, brain tumor(s), paralysis, muscular dystrophy, amyotrophic lateral sclerosis, supraoptic lateral sclerosis, primary lateral sclerosis (PLS), progressive bulbar palsy (PBP), spinal muscular atrophy (SMA), motor neuron diseases (MND), Alzheimer's disease, Parkinson's Disease.

In some embodiments, the present invention is an internal bandage device. In some embodiments, the device comprises purified cannabidiol. In some embodiments, the device comprises a cannabinoid mixture. In some embodiments, the device comprises a phytocannabinoid. In some embodiments, the device comprises an absorbable medical carrier. In some embodiments, the medical carrier comprises a gelatin foam sponge. In some embodiments, the medical carrier comprises cholesterol, polysaccharide, polyglycolide, or glycerol. In some embodiments, the medical carrier comprises seaweed and nanoparticles. In some embodiments, the medical carrier comprises steroids. In some embodiments, the medical carrier comprises hydrogel. In some embodiments, the device comprises sterile packaging. In some embodiments, the device is used to transfer CBD to the surface of an injury site. In some embodiments, the injury site is the brain. In some embodiments, the injury site is the spinal cord.

In some embodiments, the present invention is a novel method and system of treating an injury site comprising a medical carrier with at least one cannabinoid. In some embodiments, the cannabinoid is pure. In some embodiments, the at least one cannabinoid is CBD. In some embodiments, the medical carrier is absorbable in vivo. In some embodiments, the method comprises applying the medical carrier to the injury site, delivering CBD to the injury site for a sustained period. In some embodiments, CBD is delivered to the surface of an internal injury site. In some embodiments, the medical carrier conforms to the surface size of the internal injury site. In some embodiments, the injury site follows a neurological trauma. In some embodiments, the medical carrier is applied by intrathecal, subarachnoid, or epidural delivery.

In some embodiments, an absorbable bandage is saturated with CBD or multiple phytocannabinoids. In some embodiments, a bandage is saturated with CBD under sterile conditions. In some embodiments, the present invention comprises preparation of a foam mesh with CBD or other cannabinoids. In some embodiments, the present invention comprises preparing a foam mesh and saturating the mesh with cannabigerol (CBG). In some embodiments, the present invention comprises preparing a foam mesh and saturating the mesh with cannabinol (CBN). In some embodiments, the present invention comprises preparing a foam mesh and saturating the mesh with cannabidivarin, (CBV). In some embodiments, the present invention comprises preparing a foam mesh and saturating the mesh with tetrahydrocannbinol (THC). In some embodiments, the present invention comprises preparing a foam mesh and saturating the mesh with tetrahydrocannbinolic acid (THCA). In some embodiments, the present invention comprises preparing a foam mesh and saturating the mesh with cannabidiolic (CBDA) or tetrahydrocannabidivarin (THCV), or cannabchromene (CBC). In some embodiments, the present invention comprises preparing a foam mesh and saturating the mesh with cannabielsoin (CBE).

In some embodiments, the present invention is administered by open surgical procedures. In some embodiments, the present invention is administered by penetrating brain trauma procedures. In some embodiments, the present invention is administered by semi-closed surgical procedures such as in closed traumatic brain injury and guided injectable delivery. In some embodiments, the present invention is administered by intrathecal delivery. In some embodiments, the present invention is administered by epidural delivery with adjustable needle size. In some embodiments, the present invention is delivered into subarachnoid space of the brain with adjustable needle size. In some embodiments, the present invention is delivered into subarachnoid space of the spinal cord with adjustable needle size. In some embodiments, the present invention is administered in conjunction with a peripheral dosing treatment. In some embodiments, the present invention is administered before peripheral dosing treatment. In some embodiments, the present invention is administered in conjunction with a transdermal treatment. In some embodiments, the present invention is administered before transdermal treatment. In some embodiments, the present invention is administered after transdermal treatment.

Preferred embodiments of the present invention will now be described by referencing the appended figures representing preferred embodiments.

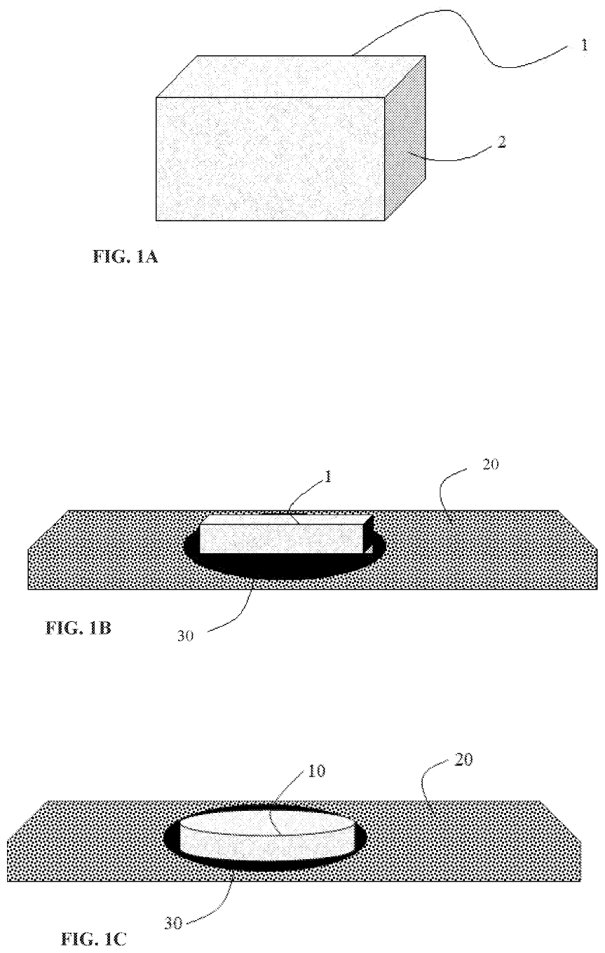

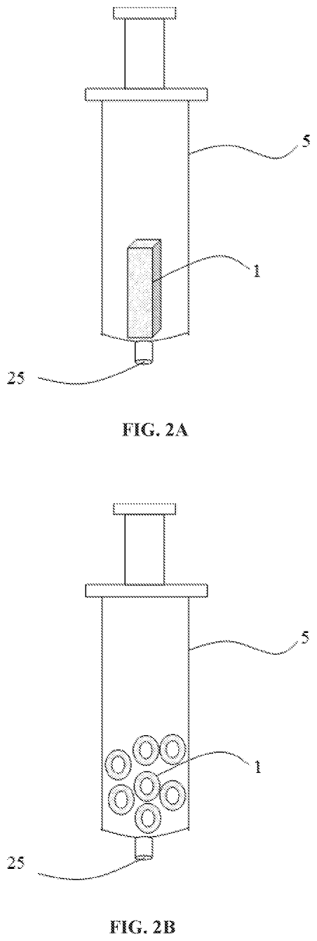

FIG. 1A depicts one embodiment of a device comprising a medical carrier 1 and at least one cannabinoid 2. In some embodiments, the at least one cannabinoid 2 is CBD. In some embodiments, the at least one cannabinoid 2 is a mixture of CBD and other phytocannabinoids or anti-inflammatory/anti-oxidant substances. In some embodiments, medical carrier 1 is saturated with at least one cannabinoid 2. The at least one cannabinoid can be applied to a shapeable and bioabsorbable bandage. The bandage can comprise a foam mesh, polymers, nanoparticles, and pellets. This device can treat an injury site and different aspects of timed events that occur after a neurological trauma. In preferred embodiments, the steps comprise: 1) Preparing a bioabsorbable medical carrier to size; 2) Preparing semi-viscous epidural/subarachnoid/intrathecal carrier vehicle; 3) Saturating the medical carrier with CBD stocks (0.1-500 mg/ml). While preferred materials for elements have been described, the device is not limited to those materials.

FIG. 1B depicts an example of applying a medical carrier 1 directly to an injury site 30 located in a user's tissue 20. In some embodiments, tissue 20 is the brain. In some embodiments, tissue 20 is the spinal cord. In some embodiments, injury site 30 is a primary injury. In some embodiments, injury site 30 is a secondary injury. In some embodiments, injury site 30 is both a primary and secondary injury. In some embodiments, injury site 30 is a result of neurotrauma.

FIG. 1C depicts a medical carrier 10 with ability to be configured into different shapes and sizes to conform to the size of injury site 30. The configuration of medical carrier 10 to injury site 30 can occur before or after the addition of at least one cannabinoid. Medical carrier 10 can comprise an absorbable gelatin sponge, nanoparticles, pellets, polymers, or other suitable materials. Configuring shapes and sizes can be performed under sterile conditions, such as with sterile scissors under a biological hood. In preferred embodiments, each of the elements of medical carrier 10 are configured together into at least one shape for immediate use, at the time of a surgery. Therefore, the order of steps to shape or combine at least one cannabinoid with medical carrier 10 is not restricted.

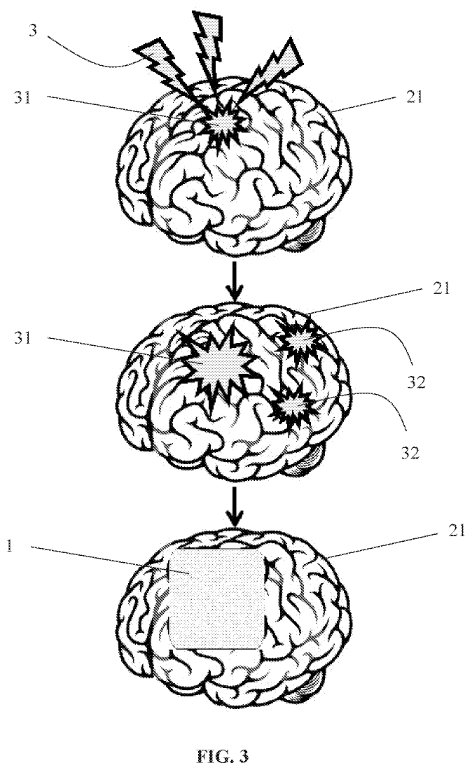

In preferred embodiments of the present invention, a medical carrier containing at least one cannabinoid, such as CBD, can be used on demand to allow direct delivery to the open surface area of a contusion or injury site, such as in the brain or spinal cord. FIGS. 2A and 2B depict examples of a delivery agent 5 and a medical carrier 1. In some embodiments, delivery agent 5 can be used to apply medical carrier 1 internally to an injury site. In some embodiments, medical carrier 1 comprises a bandage saturated with CBD. Delivery agent 5 can be used to inject medical carrier 1 subdural directly to the injection site. For example, a neurosurgeon can use delivery agent 5 after an invasive penetrating neurological trauma such as in subarachnoid hemorrhage, to inject medical carrier 1 and internally bandage the surface of affected area. Delivery agent 5 contains application site 25, where medical carrier 1 can be ejected from inside delivery agent 5 to an injury site. In some embodiments, application site 25 is further connected to an injection device such as a needle. In some embodiments, application site 25 allows for connection to various injection devices. In some embodiments, application site 25 allows for connection to various injection devices of differing sizes. In preferred embodiments, after ejection from delivery agent 5 to an injury site, medical carrier 1 will dissolve in vivo. Therefore, medical carrier 1 once injected at the desired site does not need subsequent surgical removal. Medical carrier 1 can comprise absorbable biological materials to carry CBD or other anti-inflammatory/anti-oxidant substances, including polysaccharide, polyglycolide, glycerol, gelatin, or cholesterol. In a preferred embodiment, medical carrier 1 comprises pure CBD dissolved in polysaccharide or polyglycolide. In another preferred embodiment, medical carrier 1 comprises pure CBD dissolved in cholesterol. In another preferred embodiment, medical carrier 1 comprises pure CBD dissolved in glycerol or gelatin. The presently disclosed medical carrier allows for the transfer of a biological compatible internal CBD bandage to a primary or secondary neurological injury site post-trauma. FIG. 2B shows an embodiment where delivery agent 5 contains medical carrier 1, wherein the medical carrier 1 comprises pellets or particles, such as nanoparticles.

The medical carrier 1 can be used for on demand use to allow direct neurosurgical local injection to a closed contusion site such as with a subdural hematoma. For penetrating invasive and less invasive non-penetrating contusions, CBD or other cannabinoid application can be supplemented with daily peripheral subcutaneous or oral administration using CBD as a dual treatment.

In the preferred embodiments, delivery agent 5 can be used to inject medical carrier 1 onto an injury site so that CBD or other cannabinoids will be dissolved to a low viscosity to allow absorption of medical carrier 1. This will provide for direct application to a closed contusion site such as with an epidural or subarachnoid delivery to the surface of the injury site, such as the brain or spinal cord.

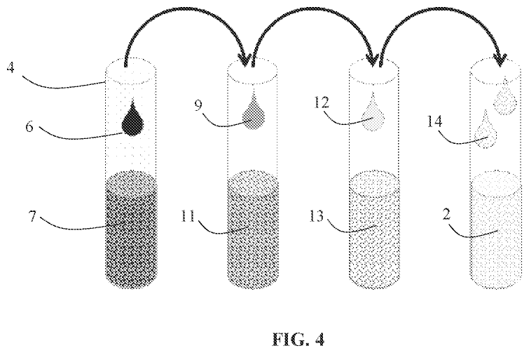

FIG. 3 depicts a method of applying a medical carrier directly to the surface of an injury site. A primary injury 31 occurs as a result of mechanical insult 3 on the surface of a tissue 21. In some embodiments, tissue 21 is the brain. A secondary enlargement of the primary contusion site 31 as well as additional secondary injury sites 32 can result from the first mechanical insult. A method of treatment comprises applying a medical carrier 1, such as an internal bandage, to at least one of the surface of primary injury 31 and secondary injury sites 32 to attenuate acute neurological injury and prevent progression of secondary neurological injury.

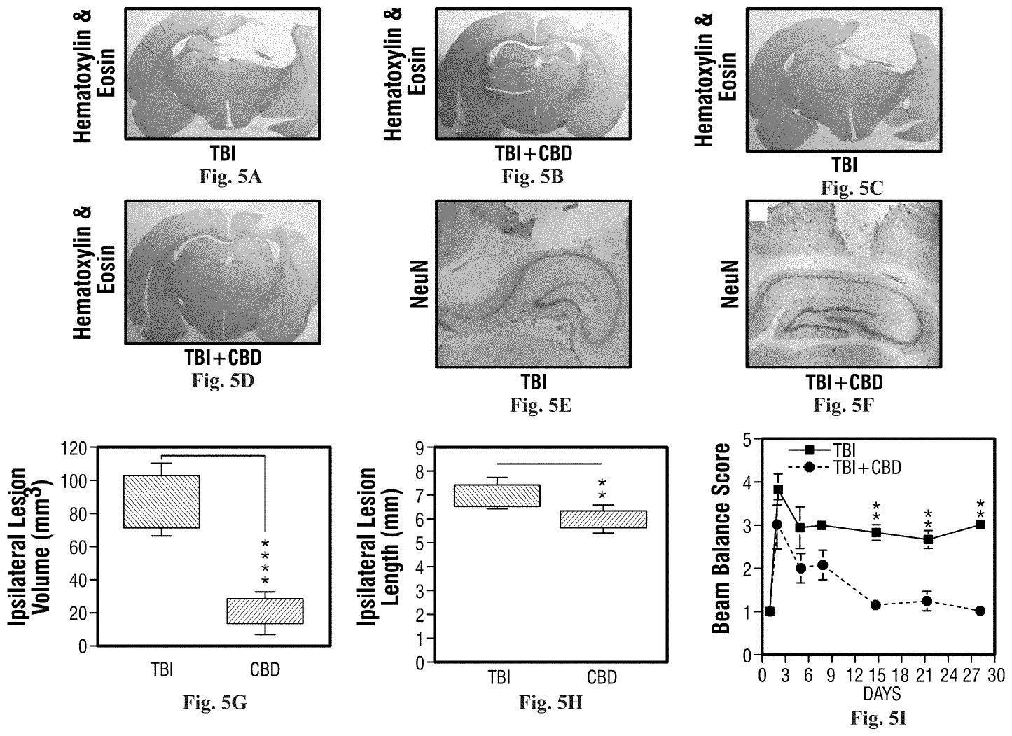

FIG. 4 depicts preparation of a cannabinoid solution for use in a medical carrier device. For example, extracted cannabinoids 6, such as CBD, is derived from marijuana leaves and stored in a bottle until use. Extracted cannabinoids 6 are not limited to a single cannabinoid but also could be a mixture of 2 or more phytocannabinoids or anti-inflammatory/anti-oxidant substances. For example, extracted cannabinoids 6 can comprise a mixture of CBD and THC, or CBD and alpha pregnanalone. In some embodiments, extracted cannabinoids 6 are pure extractions from marijuana leaves. For use in a medical carrier device, extracted cannabinoids 6 can be diluted to desired concentration(s) by mixing with solutions comprising substances with non-hazardous biological effects, such as polysaccharide or polyglycolide glycerol or cholesterol.

The present invention described herein can attenuate primary tissue injury associated with the initial insult, reduce or prevent secondary injury, as well as improve locomotor recovery. For example, an absorbable biological material pre-medicated with CBD allows the medical carrier to adhere to the contusion site and dissolve over time while continuously delivering the drug. In some embodiments, the medical carrier is administered by a dual treatment paradigm internal treatment carried out in conjunction with peripheral treatment (oral or injection). Dual treatment can simultaneously target both local and distant inflammatory processes which will reduce local swelling and primary injury as well as the spread of secondary injury. In some embodiments the dual treatment will be the internal medical carrier followed by oral administration. In some embodiments the dual treatment will be the internal medical carrier followed by intranasal delivery. In some embodiments the dual treatment will be the internal medical carrier followed by subcutaneous injection. It is surprising that dual treatment is more effective because peripheral administration should reach blood levels to deliver CBD or other anti-inflammatory/anti-oxidant drugs to the region of injury. However, the circulating dose is less efficient than direct application of the medical carrier that automates acute healing. Continued local healing occurs while systemic treatment either orally, intranasally, or by injection which treats for a longer period and maintains higher blood levels than the medical carrier alone after time passes from the insertion. Severe brain trauma cases with local bleeding are immediately addressed with a direct delivery system and safe cannabinoid treatment device. Dual treatment with central and peripheral application of CBD at the time of the injury may be most beneficial for long-term outcome.

The present invention described herein can be pre-packaged under sterile conditions in separate envelopes within one box container. These can be distributed at room temperature. In some embodiments, different shapes or sizes of the medical carrier may be pre-packaged in sterile wrapped packaging envelopes, then boxed as a source of a variety Pak of pre-shaped or unshaped bandages. Another important feature of this system is that it can accommodate other medicinal cannabinoid combinations and mix with different carriers and anti-inflammatory/anti-oxidant substances.

In some embodiments, CBD is dissolved from a high viscous to a low viscous fluid for filling a syringe to provide a less invasive delivery system of the cannabinoid. In some embodiments, an epidural/subarachnoid injection of a CBD hydrogel medical bandage device is delivered by epidural/subarachnoid injection to the contusion site of the spinal cord. The treatment can be supplemented with daily peripheral subcutaneous or oral administration of CBD as a dual treatment. The medical carrier can be pre-packaged under sterile conditions in separate vials within one box container.

While the presently provided carrier devices, methods, and systems have been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit and scope of this invention.

### Example

A pre-clinical model of cortical contusion described further herein shows that a dissolvable gelatin sponge containing CBD and applied directly to the surface of an open contusion site, then left inside to self-adsorb, causes rapid and significant reduction in lesion size compared to contusion alone. A pre-clinical model of spinal cord contusion described further herein also shows that a configured absorbable CBD saturated gelatin sponge bandage contoured to the open contusion site causes slow but significant improvement in tissue preservation and locomotor recovery over time compared to contusion alone. Dual treatment with daily supplemental subcutaneous CBD administration that follows insertion of the internal CBD bandage causes significant improvement in tissue preservation and locomotor recovery over time compared to contusion alone.

Production of CBD medical carrier. According to the invention, CBD (cannabis CO2 extracted) was obtained that was purified and concentrated at 20 mg/ml CBD and 0.3-0.8 mg/ml THC suspended in grape, coconut, or olive oil. Gelfoam sterile sponges were cut to a small cuboidal size of approximately 4.5 mm³ and then saturated overnight with the CBD stock solution in single Eppendorf tubes at room temperature.

Cortical Contusion Injury. Adult male and female Sprague-Dawley and Wistar rats (225-350 gm) received a moderately severe cortical impact injury under anesthesia with a controlled weight drop apparatus mounted on a stereotactic apparatus. Anesthesia was induced with ketamine/xylazine (60/6 mg/kg i.p.). A midline incision was performed to expose the skull. The dura was exposed with a 4.4-mm craniotomy centered at 1.4-mm posterior and 2.4-mm lateral to the bregma for placement of the 4-mm diameter foot-plate of the contusion apparatus. Contusion injury force of 600 g/cm was produced with a 20-g weight and dropping distance of 30-cm. The 20-g weight was released into a vertical guided tube that strikes the foot plate positioned at the end of the tube which is in contact with the dura. To avoid brain penetration, the foot-plate was prevented by a guide from moving greater than 2.5-mm into the dura.

Drug Administration. Rats in the TBI+CBD group received the gelfoam sponge infused with CBD (20.0 mg/ml) directly to the open contusion site overlying the dura within 2 min after the weight was removed from the apparatus. CBD was also administered systemically 10 min following surgery at 40 mg/kg followed by 20 mg/kg for all other injection days. TBI rats received gelfoam with coconut or olive oil directly onto the open contusion site overlying the dura following their respective surgical contusions. TBI rats also received vehicle, either coconut oil, olive oil, or PBS systemically at equal volumes that were administered to CBD treated animals according to weight (0.1-0.4 cc).

After direct delivery of the CBD internal gelatinized matrix, dental acrylic was used to close the wound. Animals became active 30-40 min following surgery.

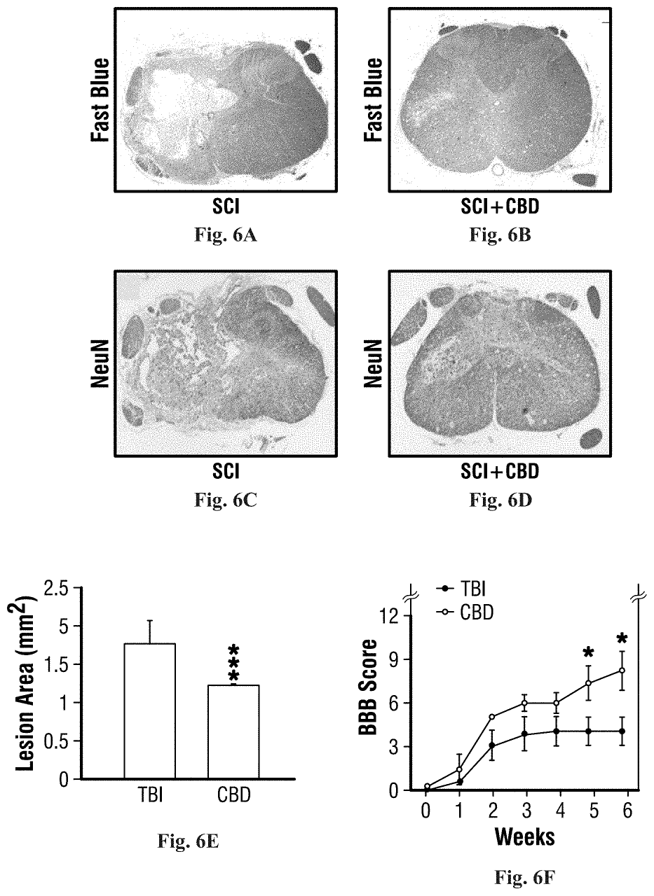

FIG. 5 depicts schematic overview perspectives of an internal CBD absorbable medical carrier applied to the internal brain wound in the rat with systemic supplementation. Hematoxylin and eosin-stained sections (3-5 μm) of contralateral and ipsilateral cortico-hippocampal injury 4 weeks after brain or TBI+CBD local and peripheral delivery system show reduced ipsilateral lesion size in young adult male and female rats.

FIG. 6 depicts schematic overview perspectives of an internal CBD absorbable medical carrier applied to the internal wound in the rat with systemic supplementation with a spinal cord contusion (SCI). Histological evaluation with Luxol Fast Blue myelin staining showed reduced volumetric lesion area with 1 week of daily CBD injections and 3 weeks of intermittent CBD treatment. Although clear sparing of spinal cord tissue was observed, the animals treated with CBD did not show comparable behavioral differences in the first 2 weeks. However, significant locomotor improvement was observed in weeks 5 and 6 with continued CBD treatment and the trend upward was already apparent in week 3 in aged female rats.

Post-injury administration of local and peripheral CBD delivery system decreased the size of the ipsilateral lesion, and the contralateral side was preserved. Box and whisker plots of ipsilateral lesion volume approximated from 12 levels of the septal caudal plane revealed marked reduction in ipsilateral cortical/hippocampal lesion volume (69.85±7.2 mm³ vs. 21.4±3.5 mm³) (N=11 vs. N=6: t=3.08, df=14, p=0.00026). Examining the area after the contusion under brightfield microscopy and scanned imaged showed the depth and width were predominantly affected at mid-anterior dorsal levels and that the CBD treatment showed protective effects at similar levels with respect to the hippocampus. Posterior levels such as Bregma AP: −6.8 or further were spared to a similar degree in the absence of CBD (1.22±0.35 mm² vs. 0.92±0.17 mm²) (p=0.347). Quantitative measurements confirmed the observations from anterior to posterior levels (Bregma AP: 1.2 to −6.8). Normalizing the data showed the area affected by the CBD treated group was 34.04±6.38% of the TBI+vehicle treated group (t=4.34, df=13, p=0.0008). Box and whisker plots also showed there was a significant reduction in total anterior to posterior lesion measurements in CBD treated animals (6.586±0.2 mm vs. 5.867±0.17 mm (t=3.04, df=14, p=0.008). Control TBI, N=10 vs. CBD, N=6, Two-tailed Student's t-Test. There was no significant correlation between the extent of the ipsilateral lesion with balance beam scores for any of the days tested following TBI+vehicle (e.g. TBI, Day 4: r=0.21; Day 14: r=0.0045) nor after TBI+CBD (CBD, Day 4: r=−0.5.22; Day 14: r=0.78).

Microscopic examination of the hippocampus after H&E staining revealed robust ipsilateral injury and moderate contralateral injury at the cellular level after TBI that was partially attenuated with the CBD local and peripheral delivery system. In the TBI+vehicle group, the contralateral hippocampus exhibited pyknotic, acidophilic stained cells with irregular shape scattered throughout the subfields with the highest amount counted in the granule cell layer (GCL) of the dentate gyrus and the least in the hilus (not illustrated). In the contralateral CAL injured cells were intermingled among healthy appearing round cell bodies. On the ipsilateral side the hippocampus was either completely missing or partly preserved exhibiting numerous shrunken, pyknotic cell bodies with varied gaps along the CA1-3 and GCL layers. In contrast, the contralateral hippocampus of animals treated with the CBD system predominantly exhibited uniform Nissl staining with round, regular shaped CA1 cell bodies that were normal in appearance however, a few injured appearing cells were scattered in the layer. In contrast to the TBI+vehicle group, the ipsilateral hippocampus was predominantly intact in all CBD treated animals but with disproportional levels of injured cells throughout the subfields. There were varied numbers of pyknotic, acidophilic, irregular shaped CA1 and CA3a (not illustrated) neurons. The granule cell layer of the dentate gyrus (GCL) exhibited a large quantity of darkly stained hyperbasophilic neurons; few scattered irregular shaped stained neurons were detected in the hilar/CA3c region following the TBI+CBD delivery system.

Since 9 of 13 animals from the TBI+vehicle group had spared ipsilateral hippocampal tissue, counting of injured vs. healthy appearing cells of the CA1 subregion was carried out in a subset of animals from the TBI+vehicle group and compared with the CBD treated group to estimate whether injury on one side is related to the amount of injury on the other side. Measurements of the CA1 area and total volumetric measurements of the ipsilateral hemisphere were used for correlating ipsilateral injury with ipsilateral and contralateral hippocampal cellular injury. Counting the ratio of ipsilateral injured CA1 neurons to healthy appearing CA1 pyramidal neurons revealed that the ratio was unchanged, however; the total number of ipsilateral CA1 injured+non-injured cells was significantly higher in the CBD treated group suggesting higher neuronal cell loss occurred in the TBI+vehicle treated group (246±18 vs. 386 60.8, t=−2.783, df=9, p=0.021).

Quantitative measurements of the contralateral hippocampus showed only the CA1 and hilar regions had lower numbers of irregular stained/injured cells compared to TBI+vehicle treated rats (CA1: 53.3±10.9 vs. 14.22±2.2; Hilus: 30.4 4.17 vs. 7.63±1.86, t=2.22, df=13, p=0.045 and p=0.002, respectively). The percent of CA1 contralateral injured cell counts was reduced by 26.7±4.37%. Although the contralateral CA1 and hilus had fewer injured cell counts in the CBD group compared to the TBI group, more stringent statistical analysis comparing the counts across the four subfields (CA1, CA3, DG, hilus) with one way ANOVA showed the difference in the amount of injured cells was not significant between the contralateral subregions for either group (TBI+vehicle, F=1.72, p=0.18; TBI+CBD, F=1.84, p=0.18). However, when comparing the hippocampi of both hemispheres, the number of injured CA1 neurons was significantly lower on the contralateral side in the TBI+CBD treated group whereas differences in cell counts were not significant in the TBI vehicle group likely due to cell loss (TBI: 91±20.8 vs. 53±10.9, p=0.1; CBD: 172.75±70 vs. 14.21±2.29, p=0.037).

FIG. 7 depicts a graphical perspective of one example of using the same local CBD treatment strategy the weight drop cortical contusion injury (CCI) model in younger adult rats. The graph shows quantified improved balance and locomotor function within a few days with CBD treatment, whereas the untreated CCI rats only improved slightly or not at all over a 2-week period. These studies appear promising for acute head trauma injuries that can lead to post-traumatic epilepsy (PTE), currently under investigation in our laboratory.

Vestibulomotor function was determined with the beam balance test in the TBI untreated and TBI+CBD treated rats. Initially, animals were tested prior to injury and exhibited balancing with steady posture on the beam, score of 1. One day after TBI, performance on the balance beam test worsened similarly in both the TBI and TBI+CBD groups. However, significant improvement in beam balance performance was observed in the TBI+CBD group beginning at day 14 through day 28 compared to TBI untreated rats (F=8.43, df=82, p=0.001. One-way ANOVA and Holm-Sidak post hoc multiple comparisons). Overall, no significant improvement in beam balance performance was observed in the TBI+vehicle and TBI untreated group from days 4-28.

The spontaneous alternating T-maze was utilized at 3 and 28 days following TBI or TBI+CBD in order to assess spatial sense, memory and cognitive ability. Naïve untreated rats spontaneously alternated between left and right arms. There was a tendency of asymmetry to the left arm, but the number of entries was not great enough to exclude the possibility that the difference was due to random sampling variability (L: 6.3±1.7 vs. R: 4.7±0.52, N=10, two-tailed t=2.1, df=18, p=0.05). At 3 days following TBI to the left hemisphere, arm entry was impaired. Asymmetry to the left was highly significant for TBI and TBI+CBD groups as the majority or all entries were made into the left arm (Two-way ANOVA, F=41.33, p<0.001). However, after 28 days, the asymmetry was reduced for CBD treated animals with a significant increase in percent alternating, whereas TBI rat's spontaneous alteration rates were unchanged (TBI: 17.5±11.8% vs. TBI+CBD: 52.5±8.53%, paired t-test: TBI: t=−1, p=0.4226 vs. TBI+CBD: t=−5.42217 p=0.01). Although CBD treated rats increased their alteration rate frequency compared to its 3 day performance, the rate did not reach control untreated levels. In addition, the time to choose right or left was unchanged and not significantly different from TBI+vehicle treated rats at 3 or 28 days post-injury ranging between 13-33 seconds.

The Novel Object Recognition test was utilized to evaluate the short-term and working memory of the TBI+CBD group compared to control TBI levels at 27-29 days and naive untreated young rats. Following habituation, animals could explore an open field with two objects for 3 consecutive days by introducing two same objects on day 1 and adding a novel object on days 2 and 3 without visual cues. Examining the distance travelled per day showed CBD treated rats increased their movement around the arena on day 2 which further increased on day 3 post-TBI compared to more vehicle treated TBI rats. The averaged distance travelled averaged from days 2+3 of testing was significantly lower after TBI (One-way ANOVA with multiple comparison method of Holm-Sidak, F=11.9, p<0.001) and after TBI+CBD (One-way ANOVA with multiple comparison method of Holm-Sidak, p=0.02). However, rats treated with our CBD delivery system travelled a greater distance than TBI+vehicle treated rats (p=0.03). While naive rats travel faster with greater speed than injured rats, CBD treated rats travelled at a greater speed than TBI rats (F=10.147, p=0.002; p=0.032, respectively). Similarly, CBD treated animals' overall mobility was greater than TBI rats and similar to naïve untreated controls. The number of episodes of interacting with old and novel objects was much greater for animals with the CBD treatment paradigm compared to TBI rats and not significantly different from naïve rats (F=16.49, p<0.001). Thus, naïve and CBD rats explored both conditioned and unconditioned (novel) objects, whereas the TBI group did not move about much and spent little or no time with old, conditioned objects or novel objects on any of the days tested. Accordingly, the time spent with the novel object was much greater for the group with the CBD delivery system compared to TBI and not significantly different from naïve rats (F=4.18, p=0.032). Comparing the progression of TBI and TBI+CBD rats time spent with the novel object on day 2 vs. day 3 showed CBD rats improved spending more time with the novel object while TBI rats did not progress (paired t-test: TBI, t=−1.47, p=0.277 vs. CBD, t=−7.83, p=0.0022).

What is claimed is:

1. An application device comprising a medical carrier, wherein the medical carrier contains at least one cannabinoid, and further wherein the medical carrier is configured for an internal injury site.

2. The application device of claim 1, wherein the medical carrier comprises at least one of gelatin, polysaccharide, polyglycolide glycerol, cholesterol, food, seaweed, nanoparticles.

3. The application device of claim 1, wherein the medical carrier is at least one of a bandage and a foam mesh.

4. The application device of claim 1, wherein the medical carrier is dissolvable in vivo.

5. The application device of claim 1, wherein the medical carrier is saturated with 0.1-500 mg/ml of CBD.

6. The application device of claim 1, wherein the medical carrier is configured to a size of 0.1-500 mm.

7. The application device of claim 1, wherein the at least one cannabinoid is cannabidiol.

8. The application device of claim 1, wherein the medical carrier conforms to the injury site.

9. The application device of claim 8, wherein the injury site is at least one of the brain and spinal cord.

10. A medical carrier device for the transfer of cannabidiol to the surface of an internal injury site, comprising:
at least one of purified cannabidiol and a cannabinoid mixture; and
an absorbable medical carrier, wherein the medical carrier is configured for internal use.

11. The medical carrier device of claim 10, wherein the absorbable medical carrier is at least one of gelatin foam sponge, hydrogel, and cholesterol.

12. The medical carrier device of claim 10, wherein the device adjusts to the surface size of the injury site.

13. A method of treating an injury site, comprising:
applying a bio-absorbable medical carrier comprising cannabidiol to an internal injury site, wherein the absorbable medical carrier adjusts to the surface size of the internal injury site.

14. The method of claim 13, wherein the injury site follows a neurological trauma.

15. The method of claim 13, further comprising treating at least one neurological disorder, wherein the at least one neurological disorder is at least one of post-traumatic epilepsy, brain tumor(s), paralysis, muscular dystrophy, amyotrophic lateral sclerosis, supraoptic lateral sclerosis, primary lateral sclerosis (PLS), progressive bulbar palsy (PBP), spinal muscular atrophy (SMA), motor neuron diseases (MND), Alzheimer's disease, and Parkinson's Disease.

16. A method comprising:
determining the dilution ratio of a cannabinoid solution;
sizing a medical carrier;
applying the solution to the medical carrier; and
applying the medical carrier to an injury site.

17. The method of claim 16, wherein the dilution ratio corresponds to at least one of injury type, injury size, age, and sex.

18. The method of claim 16, wherein the medical carrier comprises at least one of gelatin, polysaccharide, polyglycolide, glycerol, cholesterol, food, seaweed, and nanoparticles.

19. The method of claim 16, wherein the injury site is internal.

20. The method of claim 16, wherein applying the medical carrier to an injury site is at least one of a surgical procedure, epidural delivery, subarachnoid delivery, and intrathecal delivery.
